(19) United States
(12) Patent Application Publication
(10) Pub. No.: US 2006/0085018 A1
(43) Pub. Date: Apr. 20, 2006

(54) Ear comb

(76) Inventor: Rhonda Michelle Clevenger, Newport, TN (US)

Correspondence Address: Rhonda Michelle Clevenger, 1555 Restful Way, Newport, TN, 37821, US

(21) Appl. No.: 10/824,167

(22) Filed: Oct. 18, 2004

(51) Int. Cl. A61F 11/00

(52) U.S. Cl. 606/162

(57) ABSTRACT

A disposable ear cleaning appliance for insertion into an ear canal. The appliance comprises of an a long stem, which acts as a handle, that entails a cube like handling mechanism for rotating the stem, two heads that extend from each end of the stem, that act as cleaners, with two unique safety wands at the base of each head. Each safety wand is designed to guard the ear cleaning appliance from reaching the ear drum and only allow that portion of the appliance, which enters the ear canal to be capable of grasping ear wax buildup and retain the same on the appliance for removal of the earwax when the appliance is withdrawn from the ear canal. Furthermore, the fabrication and method of manufacturer of the ear cleaning appliance comprises of the use of low density polyethylene, in an injection molding process, so not to be so rigid as to be capable of doing serious damage to the ear when inserted into the ear canal.

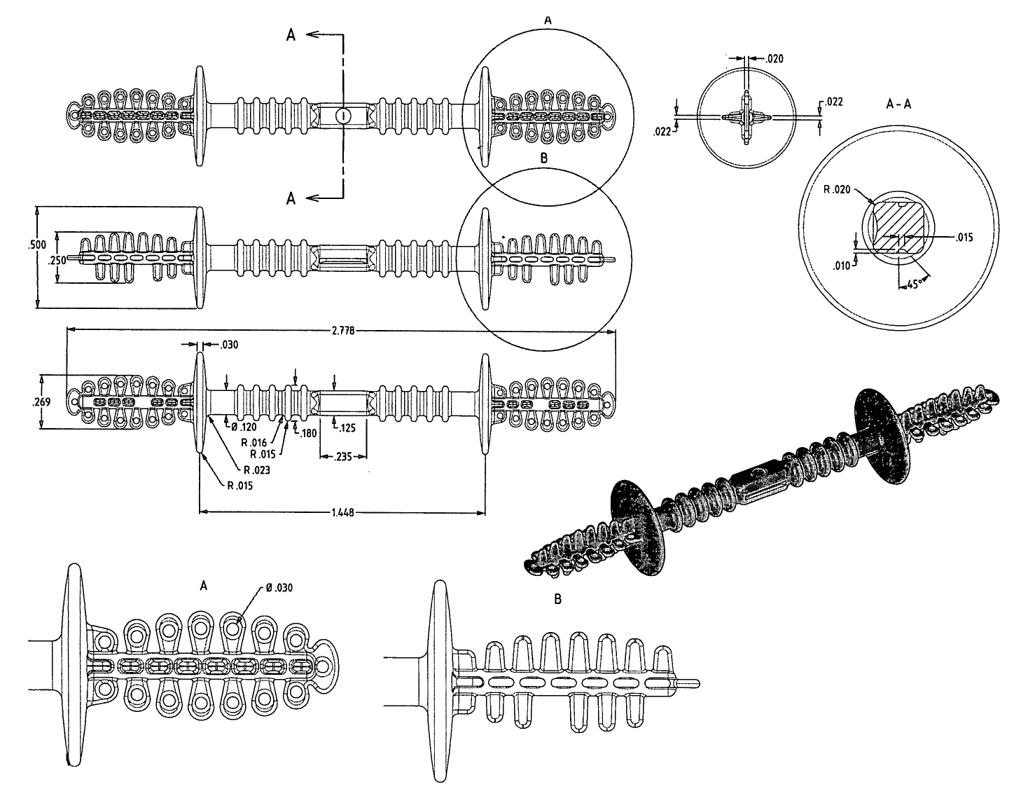

### FIELD OF INVENTION

This invention relates to ear cleaning appliances for removal of earwax from the outer ear canal.

### BACKGROUND ON INVENTION

Possibly the most commonly known ear cleaner is the appliance marketed under the tradename Q-Tip. The product comprises a wad of cotton fibers bonded onto one end of an elongated stem or handle. These products, however, suffer from multiple problems including a tendency of a user to insert the appliance too far into the ear canal and the tendency of the cotton fibers to dislodge and remain within the ear canal.

In any ear cleaning appliance, it is desired that the portion of the appliance which enters the ear canal, be readily capable of grasping any encountered matter and retain the same on the appliance for removal of the matter when the appliance is withdrawn from the ear canal.

It is desirable that the appliance not be so rigid as to be capable of doing serious damage to the ear when inserted into the ear canal. Further, desirably, the ear cleaning appliance is disposable after one use, hence its cost of production should be minimal. For this latter reason, not only the cost of the raw material employed in the appliance is of concern, but also the cost of the actual manufacture of the appliance is important.

### SUMMARY OF INVENTION

Refer to Abstract

### BRIEF DESCRIPTION OF THE DRAWING FIGURES

Fig. A is a representation of the round grasping loops and safety wand.

Fig. B is a representation of the flat grasping pads and safety wand.

Fig. A-A is a representation of the stem holding mechanism

### DETAILED DESCRIPTION OF THE INVENTION

Referring to Fig. A, a picture of the head of the stem of the appliance, which consists of an rounded open loop grasping secure cleaning head, that is adapted to enter the ear canal for removing ear wax, with an unique safety wand feature that will guard the head of the appliance from reaching the ear drum.

Referring to Fig. B, a picture of the head of the stem of the appliance, which consists of an closed pad grasping secure cleaning head, that is adapted to enter the ear canal for removing ear wax, with an unique safety wand feature that will guard the head of the appliance from reaching the ear drum.

Referring to Fig. A-A, a picture of the stem holding mechanism which can be readily grasped between the thumb and one or more fingers of the user's hand for insertion of the head into the ear canal and allow for rotation of the stem and head while the head is within the ear canal.

The ear cleaning appliance is comprised of a stem, two cleaning heads with safety wands, and a stem holding mechanism, fabricated from linier low density polyethylene. The suitable length of the stem is 2.778 mm from the end to end, the outer diameter of the stem's tube is 0.120 mm, comprising of five rings with a radius of 0.015 on each side of the holding mechanism. The holding mechanism of the stem is cubed like, with a width of 0.125 and length of 0.235. The open rounded loop grasping cleaning heads are 0.269 mm in height, at the highest point, with the inner diameter being 0.030 mm. The safety wand is 0.030 mm thick on the circumference, fanning outward to create a 0.023 radius at the intersection of the stem and a 0.015 radius around the circumference.

By reason the structure of the head and the use of linier low density polyethylene, as described hereinabove, when the head is inserted into the ear canal, each cleaning head is free to bend slightly and thereby conform to whatever surface the head may encounter within the ear canal, thereby permitting each portion of the irregular inner surface of the ear canal to be swept by rotating the stem of the appliance between the thumb and fingers of the user, using the stem holding mechanism. This action further serves to remove the matter within the ear canal and collect the same on the surface of one or more of the lengths or between, the open loop and closed pad cleaning heads, or within the interior open region of the open loop and closed pad cleaning heads of the appliance. Upon withdrawal of the appliance from the ear canal, the captured matter is likewise withdrawn from the ear canal.

Limitation of the extent of pressure of the appliance into the ear canal is provided by the user grasping the stem holding mechanism, referring to the location in A-A, hereinabove, of the stem, whereupon the thumb and finger(s) of the user is used to closely control the degree of pressure being applied to the head. More specifically, when so grasped by the user, the cubed like shaped of the holding mechanism, provides a tactile sensory location for position of the user's thumb and finger(s) when grasping the appliance and provides stability of the stem, permitting the user to sense the degree of pressure with which the head of the appliance is engaging the inner wall of the ear canal, as opposed to the situation where the stem is grasped near its end.

Limitation of the insertion of the appliance into the ear canal is provided by the safety wands, positioned below each head, referring to the location in A and B, hereinabove. The safety wands serve to limit the extent of the appliance into the ear canal by being suitably larger than the head, stem and opening of the ear canal, therefore enacting as a guard for the eardrum.

The method of manufacture of the ear cleaning appliance according to the invention, comprises of conversion of the polymer material from its normal hard granular form at room temperatures, to the liquid consistency necessary for injection at its correct melt temperature. The melt is then introduced into a die, that is sufficiently designed to provide a single-piece appliance, per specification listed hereinabove, to completely fill a cavity or cavities, by injection. When the fill is complete, heat is removed from the melt, by chilling, to convert it from a liquid consistency back to its original rigid state. The cooled ear cleaning appliance is then ejection from the mold cavity and any cores or inserts.

Whereas the dimensions of the stem and the head set forth herein as preferred, it will be understood that variations in these dimensions which do not materially alter the functioning of the appliance may be employed by a tolerance, and will be evident to one skilled in the art, given the present description of the invention.

What is claimed:

1. An appliance for ear cleaning comprising of a long stem, with two cleaning heads on each end of the stem, with a safety wand just below the heads towards the inner part of the stem, and a handling mechanism for rotating the stem, fabricated out of polymer material, using an injection molding process.

2. The appliance of claim 1 wherein said stem is of about 2.778 mm in length.

3. The appliance of claim 1 wherein said is fabricated out of linier low density polyethylene.

4. The appliance of claim 1 wherein said distance from one safety wand to the other safety wand, is of about 1.448 mm in length, from center point to center point of each safety wand.

5. The appliance of claim 1 wherein said cleaning heads are comprised of alternating rows of open rounded grasping loops and flat grasping pads.

6. The appliance of claim 1 wherein said handling mechanism is of about 0.235 in length and 0.125 in width.
